(19) United States
(12) Patent Application Publication
(10) Pub. No.: US 2015/0018754 A1
(43) Pub. Date: Jan. 15, 2015

(54) DEVICES, COMPOSITIONS AND METHODS FOR TREATING ACUTE AND CHRONIC TISSUE DAMAGE

(71) Applicant: Allan Mishra, Menlo Park, CA (US)

(72) Inventor: Allan Mishra, Menlo Park, CA (US)

(21) Appl. No.: 14/327,444

(22) Filed: Jul. 9, 2014

Related U.S. Application Data
(60) Provisional application No. 61/844,799, filed on Jul. 10, 2013.

(51) Int. Cl. A61K 35/14; A61B 17/16; A61B 17/34

(52) U.S. Cl. CPC A61K 35/19; A61B 17/3472; A61B 17/3476; A61B 17/1615; A61B 2017/564
USPC 604/22; 424/93.72

(57) ABSTRACT

A device to prepare and deliver a bioactive substance such as stem cells, platelet rich plasma, and/or other bioactive compositions. The device can utilize a hallow interior or cavity to deliver bioactive substances during a surgical procedure or to an area of cartilage defect or cartilage disorder. The treated tissues can include connective tissue, cardiac muscle or tissue, spinal tissue, internal organs, skin tissue, brain tissue, vascular tissue, ocular, ear, nose, throat tissue, and/or other hematologic, endocrine or integumentary tissue.

### CROSS-REFERENCE TO RELATED APPLICATIONS

This application claims the benefit of priority under 35 U.S.C. §119(e) from U.S. Provisional Application No. 61/844,799, filed Jul. 10, 2013, entitled “DEVICES, COMPOSITIONS AND METHODS FOR TREATING ACUTE AND CHRONIC TISSUE DAMAGE” and is hereby incorporated herein by reference in its entirety.

### BACKGROUND

1. Field of the Invention

The field of the invention relates to repair or restoration of articular cartilage in general but could be applied to a variety of other tissues. A specific set of devices to prepare and deliver bioactive substances such as stem cells, platelet rich plasma, and/or other bioactive agents or compositions are described.

2. Description of the Related Art

Current clinical treatments for symptomatic cartilage defects involve techniques aimed at: 1) removing surface irregularities by shaving and debridement 2) penetration of subchondral bone by drilling, fracturing or abrasion to augment the natural repair response 3) joint realignment or osteotomy to use remaining cartilage for articulation 4) pharmacological modulation 5) tissue transplantation and 6) cell transplantation (Newman, 1998; Buckwalter and Mankin, 1997). Most of these methods have been shown to have some short term benefit in reducing symptoms (months to a few years) while none have been able to consistently demonstrate successful repair of articular lesions after the first few years. There is a need for a technique and/or procedure that can demonstrate long term successful repair of articular lesions and/or cartilage or other tissue defects.

### SUMMARY

In one aspect, a method of treating an injury, wear or defect in an individual comprising: identifying an area of injury, wear or defect; inserting a device having an awl shaped distal end into the identified area; creating a cavity in the area using the awl shaped distal end of the device; attaching a delivery system to an access port for delivery of a bioactive substance; delivering the bioactive substance through the internal channel and into the identified area; closing the access port on the device; compressing the bioactive substance into the identified area; and removing the device. In some embodiments, the method further comprising repeating steps (b) through (h). In some embodiments, the method wherein the area of injury, wear or defect comprises a tissue selected from the group consisting of connective tissue, cardiac muscle or tissue, spinal tissue, internal organs, skin tissue, brain tissue, vascular tissue, ocular, ear, nose, and throat tissue. In some embodiments, the method wherein the spinal tissue is selected from the group consisting of nerves, spinal cord, disc material and vertebral bodies. In some embodiments, the method wherein the internal organ is selected from the group consisting of pancreas, lungs, liver, intestines, and bladder. In some embodiments, the method wherein the vascular tissue is selected from the group consisting of veins, arteries and lymphatic tissue. In some embodiments, the method wherein the connective tissue is selected from the group consisting of articular cartilage, meniscus cartilage, ligament, tendons, fascia, bone and spinal tissue. In some embodiments, the method wherein the bioactive substance is selected from the group consisting of platelet rich plasma, stem cells, bone marrow cells, bone marrow aspirate, drugs, individual growth factors and synthetic materials. In some embodiments, the method wherein the bioactive substance is platelet rich plasma, wherein no exogenous activator is added to the PRP prior to delivery into the identified area. In some embodiments, the method wherein the PRP comprises platelets obtained from the individual. In some embodiments, the method further comprising the step of titrating the PRP to obtain a pH of about 7.3 to 7.5, wherein the titration is performed using a bicarbonate buffer. In some embodiments, the method further comprising the step of mixing the PRP substantially simultaneously prior to delivery into the identified area, with one or more ingredients selected from the group consisting of thrombin, epinephrine, collagen, calcium salts, and pH adjusting agents.

In another aspect, a device comprising a self-tapping drill bit, the drill bit comprises: a middle section without flutes, the middle section comprising an internal channel disposed along a longitudinal axis within the drill bit; and a tapered end; wherein the internal channel comprises a hollow length extending along at least a part of the longitudinal axis, the hollow length having one or two inputs and at least one output at the tapered end; and wherein the at least one opening allows for the release of a bioactive substance. In some embodiments, the device wherein the at least one output comprises multiple openings. In some embodiments, the device wherein the hollow length extends along the entire length of the longitudinal axis. In some embodiments, the device wherein the internal channel is adapted for delivery of a liquid. In some embodiments, the device wherein the bioactive substance is selected from the group consisting of platelet rich plasma (PRP), stem cells, bone marrow cells, bone marrow aspirate, drugs, individual growth factors and synthetic materials. In some embodiments, the device further comprising an awl, wherein the awl is configured to be used in combination with the drill bit and introduced into or around the area of where the drill bit is used.

In another aspect, composition for use during an osteotomy procedure comprising: at least one of a bone supporting substitute, wherein the bone supporting substitute comprises calcium phosphate; and at least one bioactive substance selected from the group consisting of platelet rich plasma, vitamin D, stem cells, bone marrow cells, bone marrow concentrate, bone marrow aspirate, or adipose concentrate; and wherein the composition helps to support an osteotomy and affect a change in the alignment of bone. In some embodiments, the composition wherein the bone comprises a femur or a tibia.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows a prototype of a straight, cannulated MicroPik.

FIG. 2 shows a prototype of a curved, cannulated, MicroPik with portal for injection of bioactive material.

FIG. 3 illustrates a schematic of an embodiment of a cannulated MicroPik with a portal.

FIG. 4 illustrates a schematic of an embodiment of a schematic of cannulated MicroPik.

FIG. 5 illustrates a schematic of an embodiment of a schematic of cannulated MicroPik.

FIG. 6 illustrates an embodiment of a MicroPik inserted into articular cartilage defect.

FIG. 7 shows a biologic chondroplasty (BioChondroplasty).

FIG. 8 shows a biologic high tibial osteotomy (BioHTO).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The bone marrow-stimulation techniques of shaving, debridement, drilling, fracturing and abrasion athroplasty permit temporary relief from symptoms but produce a sub-functional fibrocartilagenous tissue that is eventually degraded. Pharmacological modulation supplying growth factors to defect sites can augment natural repair but to date insufficiently so (Hunziker and Rosenberg, 1996; Sellers et al., 1997). Allograft and autograft osteochondral tissue transplants containing viable chondrocytes can effect a more successful repair but suffer from severe donor limitations (Mahomed et al., 1992; Outerbridge et al., 1995).

One specific example of a cartilage disorder is a full thickness defect in the articular surface of a knee. This could occur with an acute injury or via chronic degeneration. Many different non-operative and operative treatments exist for this problem. Typically, rest, icing, physical therapy and some form of oral or injected medication is recommended. Braces and alternative forms of treatment such as acupuncture may also be tried. When these fail, surgery is offered as an option. The spectrum of surgical treatment options include: arthroscopic debridement and lavage, osteotomy, or total knee replacement. Another option is micro-fracture surgery.

Micro-fracture surgery involves using a solid awl to penetrate the subchondral bone that overlies a cartilage defect. The awl can create a hole or cavity in the bone that leads to the bone marrow underneath the cartilage defect. Repair of the cartilage defect using this technique can be accomplished via the cells that leak out of the bone marrow. These bone marrow cells include bone hematopoetic and mesenchymal stem cells. These cells have the ability to transform into a variety of cell types including cartilage.

Micro-fracture surgery has been shown to be inconsistently efficacious. Successful results rely on a complete fill of the defect with a significant number of cells. If the fill of the defect is inadequate, the clinical results are typically either equivocal or even poor. The long term results especially in athletes is not ideal.

This surgery, however, is technically easy to perform so if a way were to be developed to enhance these methods it would be of benefit to both patients and orthopedic surgeons. There is a need for a way to improve the ability of the body to fill that defect. If there is better defect fill, better clinical outcomes are expected. In some embodiments described herein, the techniques and procedures can be used to improve the filling of the cartilage defect.

While the described embodiment represents a preferred embodiment of the present invention, it is to be understood that modifications will occur to those skilled in the art without departing from the spirit of the invention. The scope of the invention is therefore to be determined solely by the appended claims.

In one embodiment, the device is a device to deliver bioactive materials to an area of damaged cartilage in order to produce a better repair. The treated tissue may be connective tissue, cardiac muscle or tissue, spinal tissue (including nerves, spinal cord, disc or vertebral bodies), internal organs (including pancreas, lungs, liver, intestines, bladder or other solid organ tissue), skin tissue, brain tissue, vascular tissue (including veins, arteries or lymphatic tissue), ocular, ear, nose or throat tissue, or other hematologic, endocrine or integumentary tissue. In an embodiment, the tissue is connective tissue. Connective tissue includes but is not limited to cartilage (articular and meniscus), ligament, tendons, fascia, bone and spinal tissue.

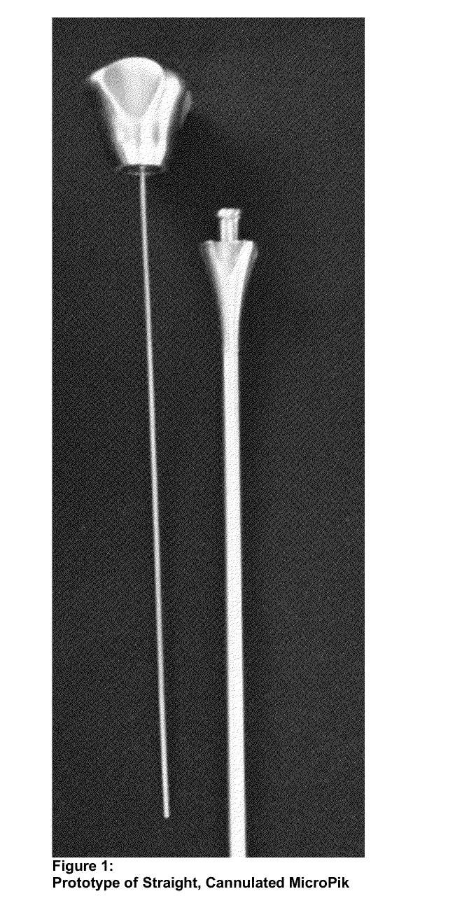

In some embodiments, the device comprises a cannulated, fenestrated awl for use to augment micro-fracture surgery. FIG. 1 illustrates an embodiment of the awl. The awl body extends along a generally longitudinal axis from a proximal end of the handle to an awl tip at a distal end of the shaft. The handle may be configured to be grasped by one or more hands of a user in order to maneuver the shaft during a surgical procedure. The handle may be any convenient shape including but not limited to round, rectangular, or polygonal. In some embodiments, the handle includes a textured and/or contoured surface to provide a suitable grip for the user. The handle may have a length in a range from about 2-24 inches, more specifically 3-8 inches. The tip of the “MicroPik” may be fashioned to be disposable and screwed into a non-disposable shaft as one of many potential options. The tips may also be made in various angles as needed from 0-180 degrees. The tips would be made to a sharp point with fenestrations around that point to inject bioactive materials.

While the described embodiment represents an embodiment of the present invention, it is to be understood that modifications will occur to those skilled in the art without departing from the spirit of the invention. The scope of the invention is therefore to be determined solely by the appended claims.

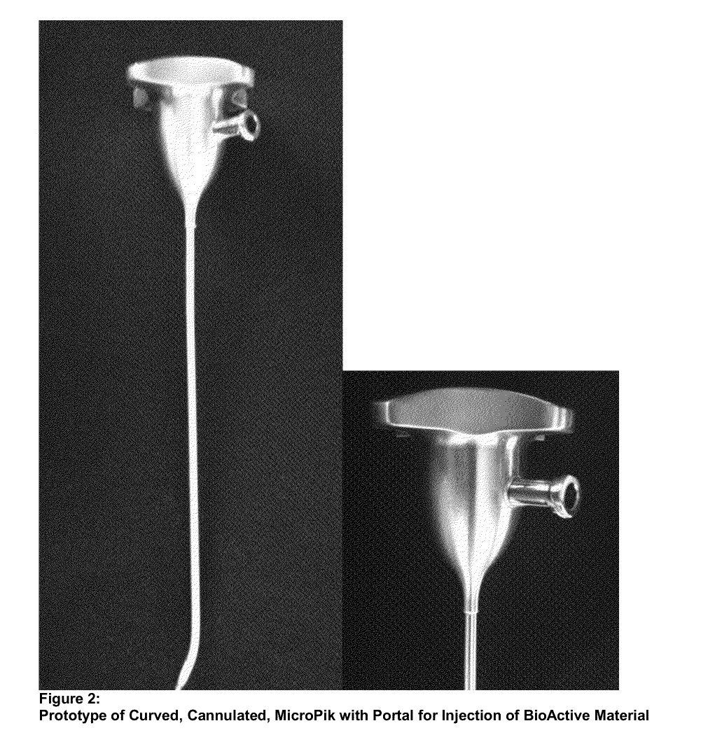

In one embodiment, the device is a device to deliver bioactive materials to an area of damaged cartilage in order to produce a better repair. FIG. 2 illustrates an embodiment of a prototype of a curved, cannulated, MicroPik with a portal for injection of bioactive material. The treated tissue may be connective tissue, cardiac muscle or tissue, spinal tissue (including nerves, spinal cord, disc or vertebral bodies), internal organs (including pancreas, lungs, liver, intestines, bladder or other solid organ tissue), skin tissue, brain tissue, vascular tissue (including veins, arteries or lymphatic tissue), ocular, ear, nose or throat tissue, or other hematologic, endocrine or integumentary tissue. In some embodiments, the tissue is connective tissue. Connective tissue includes but is not limited to cartilage (articular and meniscus), ligament, tendons, fascia, bone and spinal tissue.

In another embodiment, the awl may be used to deliver growth factors such as platelet-rich plasma, to the scalp to stimulate hair growth.

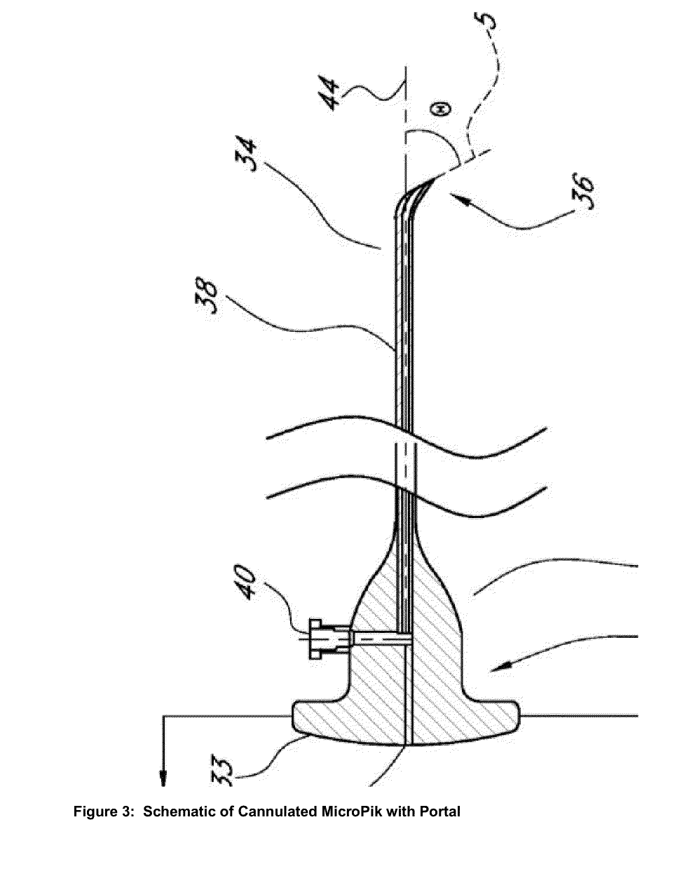

In some embodiments, the device comprises a cannulated, fenestrated awl for use to augment micro-fracture surgery. FIGS. 3, 4 and 5 schematically illustrate an embodiment of the awl that comprises an awl body having a handle and a shaft. The awl body extends along a generally longitudinal axis from a proximal end of the handle to an awl tip at a distal end of the shaft. The handle may be configured to be grasped by one or more hands of a user in order to maneuver the shaft during a surgical procedure. The handle shown in FIG. 1 is depicted as square-shaped but may be any convenient shape including but not limited to round, rectangular, or polygonal. In some embodiments, the handle includes a textured and/or contoured surface to provide a suitable grip for the user. The handle may have a length in a range from about 2-24 inches, more specifically 3-8 inches.

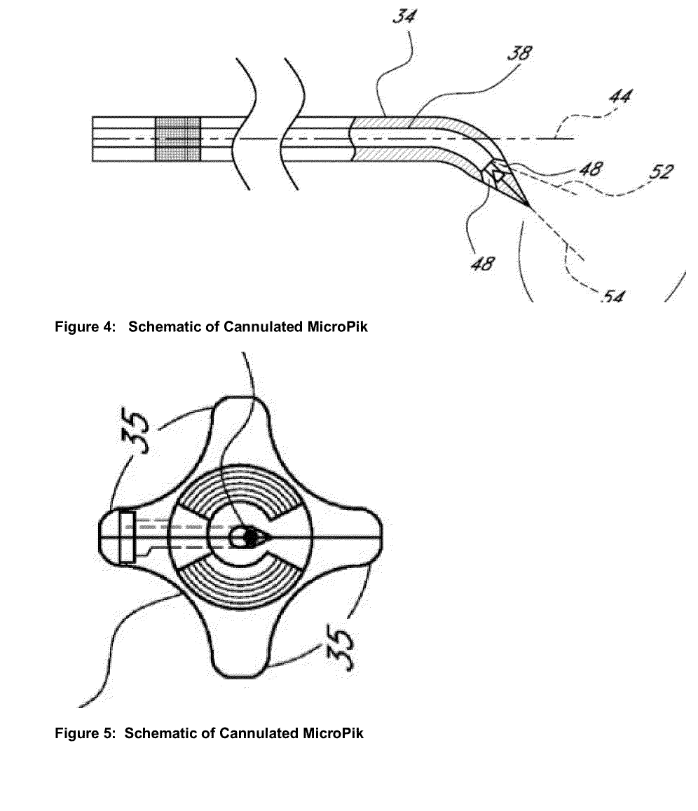

The shaft extends from a distal end of the handle to the awl tip. The shaft may be any convenient length but specifically the shaft can be from 2-24 inches and about 5-12 inches. The shaft may be substantially straight as shown, for example, in FIG. 1. In some embodiments, at least a portion of the shaft is curved as shown in the embodiments in FIGS. 4 and 5. The shaft may have any suitable cross-sectional shape such as, for example, circular, oval, polygonal, etc. As schematically shown in FIG. 1, the shaft may have a transverse width that tapers from a proximal end of the shaft 14 towards the awl tip. In other embodiments, the transverse width of the shaft 14 is substantially uniform away from the awl tip. Importantly, the tips as previously described may be detachable from the shaft and may be made to be disposable with or without the shaft.

In some embodiments, the awl body is formed as a substantially integral unit. In other embodiments, the handle and the shaft 14 are formed separately and then attached to each other. For example, the handle and the shaft may be attached by welds, adhesives, and/or mechanical coupling (e.g., mutually engaging threaded portions). The awl may be made out of any suitable material(s) including but not limited to metal, bone, ceramic, plastic or rubber or any number of potential materials based on the tissue that needs to be treated. In an embodiment, the awl shaft is constructed of a biocompatible material such as a metal, and the awl handle is constructed of hard plastic. In some embodiments, the awl can be sufficiently durable so that the awl can be manipulated to facilitate penetration of tissues such as cartilage and/or bone. In some embodiments, some or all of the awl is made of silver. Silver is used for its anti-microbial action and may be useful especially in cases where the rate of infection for a procedure may be high or the patient population is at high risk. For example, the awl may be formed from sufficiently hard and durable materials that the awl substantially resists deformation when placed under tensile and/or compressive forces (e.g., when struck with a mallet during a surgical procedure). In some embodiments, the awl is disposable. Alternatively, selected parts of the awl may be disposable such as, for example, the entire shaft including the tip and/or the tip alone. In embodiments where the shaft (or portions thereof) are disposable, the shaft advantageously may be configured to be readily attached and/or detached from the handle such as by use of a threaded portion that can engage a complimentary threaded recess in the handle.

In some embodiments, the awl is cannulated. For example, at least a portion of the awl body may include a lumen. In some embodiments, the lumen extends from the proximal end of the handle to the awl tip. The lumen may be disposed substantially along the longitudinal axis of the awl body. The lumen has a cross-sectional area, which may be substantially uniform along the length of the lumen. In some embodiments, the cross-sectional area of the lumen may vary along the length of the lumen, for example, by decreasing toward the awl tip. The shape of the cross-section of the lumen may be substantially circular, oval, polygonal, or any other suitable shape. In an embodiment, the lumen is substantially circular in cross-section and has a diameter in a range from about 1 mm to 20 mm or more. In some embodiments, the awl includes two or more lumens.

The cannulated interior may optionally include a removable insert such as a plunger to push the material down the interior shaft of the device (not shown). In some embodiments, the awl includes an attachment point for a syringe or catheter that may be used to transfer a fluid (such as a bioactive material) through the lumen. The attachment point may be open or closed by use of, for example, a removable plug. As illustrated in FIG. 1, the attachment point may be disposed on the proximal end of the handle. In other embodiments, the attachment point may be disposed elsewhere on the awl such as, for example, along a side of the handle or along the shaft. In some embodiments, two or more attachment points may be used. In certain embodiments, the lumen extends from the attachment point to the awl tip.

The distal end of the lumen may include one or more openings at and/or near the awl tip to permit fluid to flow from the tip. In an embodiments, the awl tip may have multiple openings, including two, three, four, five, six openings, or more than six openings. In some embodiments, the amount of openings is only limited by the available space on the awl tip to allow for openings of the specified dimensions to perform the specified tasks. In some embodiments, the awl tip can be perforated like a sieve (not shown). In some embodiments the awl tip has a single opening. The openings may be formed at the distal end of the awl tip and/or along the sides of the shaft near the tip.

The awl tip may be shaped to enable various functionalities. For example, the awl tip may be pointed or may be flat-bladed like a scraper. The shape of the awl tip advantageously may have any shape that is capable of creating a cavity in tissue suitable for introduction of a fluid material. The awl tip may be configured to be removable from the distal end of the shaft. For example, the awl tip may comprise a threaded portion configured to engage a complimentary threaded portion at the distal end of the shaft.

In one embodiment, the awl includes a handle and a shaft terminating at an awl tip. In this embodiment, the proximal end of the handle comprises a knob that facilitates movement of the awl. The knob may have a size and shape that permit the awl to be easily grasped and manipulated so that the tip may be inserted into tissue. For example, the awl may have four transverse prongs. In other embodiments, two, three, five, six, or more prongs may be used. As discussed the awl tip may be shaped, e.g., pointed or flat bladed like a scraper. In some embodiments, any tip capable of creating a cavity for introduction of a fluid material can be used. A dial on the handle could also be used to adjust the tip angle.

The awl may be cannulated. For example, one embodiment comprises a lumen that extends from a side port to the awl tip. The side port may be configured to engage a syringe and/or a catheter for example via a luer lock. Fluid, such as a bioactive material, may be introduced through the side port and may exit through one or more openings at or near the awl tip. In some embodiments, the lumen additionally or alternatively extends through the handle to an attachment point at the proximal end of the handle, which may be configured to engage a syringe and/or a catheter (e.g., via a luer lock). In such embodiments, fluids may be introduced at the attachment point and/or the side port. Fluids introduced at the attachment point and the side port may comprise the same or different material.

In some embodiments, the awl tip may be angled at an angle θ with respect to a longitudinal axis of the shaft. The angle θ may be fixed or variable in different embodiments. The angle θ may be any convenient angle, between 0°-120° or between 30°-60°. The lumen is disposed substantially along the longitudinal axis of the shaft and is angled to join the tip. The distal end of the lumen terminates in two openings that permit output of fluid at the surgical site. In other embodiments, a different number of openings may be utilized such as, for example, one, two, three, four, five, six, or more. As described above, in some embodiments, the tip may be perforated (like a sieve). The openings may define an exit path for the fluid that flows through the lumen. The fluid exit path may be angled with respect to awl tip axis. The angle between exit path and tip axis may be in a range from about 0°-45° and is about 30° in one embodiment. Other angles between 0° and 180° may be used.

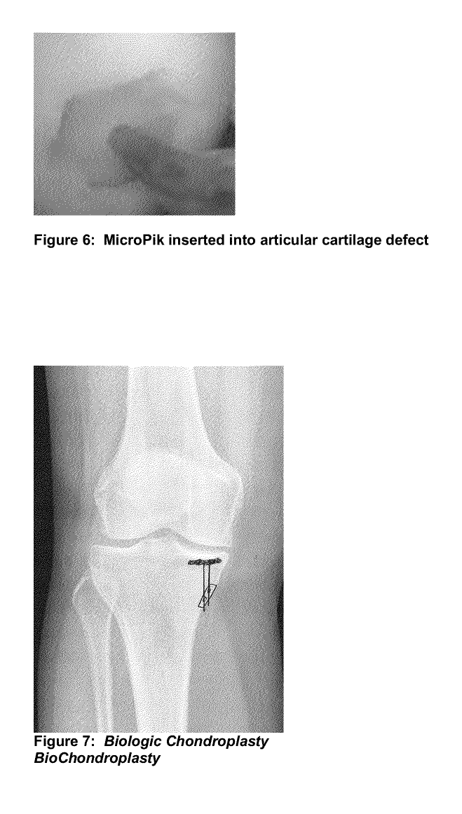

The tissue may be selected from one of the follow types but is not limited to these specific tissues: connective tissue, cardiac muscle or tissue, spinal tissue (including nerves, spinal cord, disc or vertebral bodies), internal organs (including pancreas, lungs, liver, intestines, bladder or other solid organ tissue), skin tissue, brain tissue, vascular tissue (including veins, arteries or lymphatic tissue), ocular, ear, nose or throat tissue, or other hematologic, endocrine or integumentary tissue. In an embodiment, the tissue is connective tissue. Connective tissue includes but is not limited to cartilage, ligament, tendons, bone and spinal tissue. One tissue could be damaged articular cartilage of the knee, hip, shoulder, ankle, elbow wrist, hand, foot or spine. FIG. 6 illustrates an embodiment of a MicroPik inserted into an articular cartilage defect.

The tissue may be human or other non-human animal tissue. In some embodiments, the device can be used in human and animal patients, particularly veterinary animals such as dogs, cats, horses, pigs, sheep, and cows. In other embodiments, the device can be used on a human subject.

In an embodiment, the device may be used to treat a full or partial thickness cartilage injury inside a joint in a human or animal. The device can be specifically and uniquely designed to initially penetrate tissue and then be able to deliver a bioactive substance into that area of penetration.

Any bioactive substance can be delivered via the device. These may include but are not limited to platelet rich plasma (PRP), stem cells, bone marrow cells, bone marrow aspirate or concentrate, drugs such as small molecules, individual growth factors or synthetic materials. These may also be delivered alone or in combination.

In an embodiment, the bioactive substance may include a biocompatible composition that comprises unactivated platelets, activated platelets, platelet releasate(s), and/or other bioactive substances known in the art. In some embodiment, the bioactive substance comprises platelet-rich plasma (PRP).

PRP is an enriched platelet-containing mixture, isolated from whole blood, which is resuspended in a small volume of plasma. While whole blood may contain about 95% red blood cells, about 5% platelets and less than 1% white blood cells, PRP may contain 95% platelets with 4% red blood cells and 1% white blood cells. PRP can be combined with activating agents such as thrombin or calcium which activate the platelets to release their contents such as cytokinins and other growth factors. In some embodiments, PRP is used without activation.

The term “PRP” as used herein is a broad term which is used in its ordinary sense and is a concentration of platelets greater than the peripheral blood concentration suspended in a solution of plasma, with typical platelet counts ranging from 500,000 to 1,200,000 per cubic millimeter, or even more. PRP is formed from the concentration of platelets from whole blood, and may be obtained using autologous, allogenic, or pooled sources of platelets and/or plasma. PRP may be formed from a variety of animal sources, including human sources.

Platelets are cytoplasmic portions of marrow megakaryocytes. They have no nucleus for replication; the expected lifetime of a platelet is some five to nine days. Platelets are involved in the hemostatic process and release several initiators of the coagulation cascade. Platelets also release cytokines involved with initiating wound healing. The cytokines are stored in alpha granules in platelets. In response to platelet to platelet aggregation or platelet to connective tissue contact, as would be expected in injury or surgery, the cell membrane of the platelet is “activated” to secrete the contents of the alpha granules. The alpha granules release cytokines via active secretion through the platelet cell membrane as histones and carbohydrate side chains are added to the protein backbone to form the complete cytokine. Platelet disruption or fragmentation, therefore, does not result in release of the complete cytokine.

A wide variety of cytokines are released by activated platelets. Platelet derived growth factor (PDGF), transforming growth factor-beta (TGF-b), platelet-derived angiogenesis factor (PDAF) and platelet derived endothelial cell growth factor (PD-ECGF) and insulin-like growth factor (IGF) are among the cytokines released by degranulating platelets. These cytokines serve a number of different functions in the healing process, including helping to stimulate cell division at an injury site. They also work as powerful chemotactic factors for mesenchymal cells, monocytes and fibroblasts, among others. For the purposes of this patent, the term “releasate” refers to the internal contents of the platelet, including cytokines, which have the potential to affect another cells' function.

In some embodiments, an activator is not used with PRP as the bioactive substance. Collagen, a major component of connective tissues, is a strong activator of platelets. Thus, when the inventive platelet composition is introduced into and/or around connective tissue, platelets in the platelet composition may bind to the collagen and then be activated. This reduces or eliminates the need for administering an exogenous activator such as thrombin. The disadvantages of thrombin use have been noted above. Other strong activators, such as calcium ions, can cause severe pain, unintentional clotting, and other undesirable side effects. Thus, in an embodiment, PRP can be used as the bioactive substance with no or substantially no exogenous activator added, or in the preparation of the bioactive PRP composition. Of course, exogenous activators may still be employed if a physician determines that they are medically necessary or desirable.

In some embodiments, growth factors or growth factor inhibitors, small molecule pharmaceuticals such as NSAIDS, steroids, and anti-infective agents may be introduced using the microfracture awl device.

The description herein may be applied to any tissue. An area of a cartilage defect would initially be identified by x-ray, MRI, ultrasound or other imaging modality. The imaging study used is determined by the tissue type. Commonly used imaging methods include, but are not limited to MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging and ultrasound techniques. The patient may also assist in locating the site of tissue injury or damage by pointing out areas of particular pain and/or discomfort. Other ways of finding the area of the defect include but are not limited to direct visual inspection and palpation.

Once the area of the cartilage defect has been identified, the device would be inserted into that area either under direct vision or via robotic or imaging guidance. Once inside the lesion area, it would be used to create a cavity. In the case of the knee, this would be a cavity in the subchondral bone leading directly to the bone marrow. A syringe or other delivery system would then be attached to the cannulated device to directly deliver bioactive substances into the cavity to affect a change in the tissue.

In the case of the cartilage defect, one possible bioactive substance is PRP. In some embodiment, other substances such as drugs, small molecules, viruses for gene therapy or other substances are applied to the cavity by the inventive device. In some embodiments, the device is used multiple times in a specific defect to create multiple cavities and repeatedly deliver these bioactive substances.

The device may optionally include a plunger to put through the cannula to push the bioactive material into the cavity created by the awl.

Importantly, it has been found by the inventor that platelet rich plasma (PRP) delivered to mesenchymal stem cells increases their proliferation and leads them toward chondrogenic differentiation. Using this inventive device, PRP could be delivered to the defect and lead to improved fill and therefore better clinical outcomes. Specifically, PRP has been shown to increase mesenchymal stem cell proliferation fivefold in seven days. Cartilage markers such as aggrecan and SOX-9 are significantly increased in the presence of PRP.

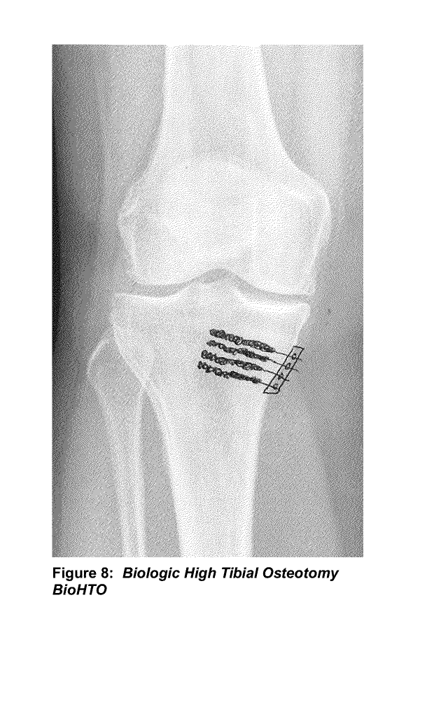

Importantly, the device described herein could be used in a antegrade or retrograde fashion alone or in combination with the devices and procedures described in the following description. The awl describe herein could be used alone or in combination with a flexible drill bit that would be cannulated. In an application, the awl would be used to access the area of damaged cartilage in a retrograde fashion in a biologic chondroplasty (biochondroplasty) procedure as shown in FIG. 7. The sizes of the drill could be from 0.5 mm to 10 cm or more. A set of drill bits can be 1 mm, 2 mm, 4 mm, 6 mm, 8 mm, and 10 mm. The microfracture awl device would then be introduced into and or around the area of where the drill had been or it could be used alone. After accessing the area of damage, the awl and or drill would be used to either aspirate and/or inject bioactive substances into the area as shown in FIG. 7. These substances could be a variety of substances including but not limited to platelet-rich plasma, bone marrow concentrate, stem cells of any kind, lipoaspirate or concentrate. They could be used alone or in combination with small molecule drugs and or known substances such as calcium phosphate, vitamin D (in any form) and or metals such as silver. In an embodiment, silver would be used to enhance or improve the anti-microbial action of the injected or prepared materials. Specifically, silver in minute or any quantity may combined with stem cells, platelet-rich plasma or other materials as another composition. Sealants such as thrombin and or calcium or others could be used before, during or after application of the bioactive substances.

One specific composition would be platelet-rich plasma containing platelets in a concentration of about 500,000/uL to about 7,000,000 in combination with silver, silver nanoparticles or silver ions in concentrations ranging from 0.01 ug/L to 100 ug/L or higher. The silver concentration could also be from 100 ug/L to 100,000 ug/L or higher. The platelet rich plasma could also contain white blood cells and specifically monocytes, lymphocytes and neutrophils in a variety of concentrations. The concentration of monocytes could be from 0 to 100,000/uL or higher. The concentration of lymphocytes could be from 0 to 100,000 or higher. The concentration neutrophils could be from 0 to 100,000 or higher. The overall concentration of white blood cells could also be from 0-100,000 or higher. This composition could be used alone or also be combined with calcium phosphate or other materials to create a spacer or areas of infection. One specific application of such a composition would be to create a spacer after to help treat an infected total knee, hip or shoulder replacement. The biochondroplasty procedure would consist of identifying an area of a chondral defect anywhere in the body via x-ray, CT, MRI, direct visualization, or other methods. The area would then be prepared with the devices describe above to create an improved chondral surface and/or improve patient outcome. Specifically, as an example, the proximal tibial plateau would be identified and then via fluoroscopic guidance and a small minimally invasive approach, the cannulated awl describe herein would be introduced into and around the defect penetrating the subchrondral zone and then a bioactive substance or substances would be introduced to enhance or improve the cartilage and surrounding bone. Support of the underlying subchondral bone may be done at the same time before or after the biochondroplasty.

The biologic high tibial osteotomy procedure (BioHTO) could be done alone or in combination with the biochondroplasty. FIG. 8 shows a biologic high tibial osteotomy procedure (BioHTO). This procedure would use similar tools to the ones described above but could involve the injection of calcium phosphate or other bone supporting material alone or in combination with platelet rich plasma, vitamin D, stem cells, bone marrow or bone marrow concentrate, adipose concentrate. The composition of bone supporting material and bioactive substances can be used to help to support an osteotomy and affect a change in the alignment of the bone. In some embodiments, the bone supporting material can include calcium phosphate. In some embodiments, the bioactive substances can include platelet rich plasma, vitamin D, stem cells, bone marrow or bone marrow concentrate, adipose concentrate. In some embodiments, the components of the bone supporting material can also act as the bioactive substance. In some embodiments, the bioactive substance can act or be used in place of the bone supporting material. In some embodiments, the composition can include the use of calcium phosphate or other bone supporting substitute during a biologic osteotomy procedure to affect a change in the overall alignment of the knee or leg. In some embodiments, the composition can include calcium phosphate, platelet rich plasma, vitamin D, stem cells, bone marrow, bone marrow concentrate, or adipose concentrate in any combination to help support an osteaotomy and affect a change in alignment of a bone. In some embodiments, the osteotomy procedure and the change in alignment of the bone can be in the knee or leg. In some embodiment, the bones can be the femur or the tibia. In some embodiments, the components and compositions as described herein can be assembled into a kit to perform and/or create the methods, procedures, and compositions as described herein.

Multiple drill holes would be made via a minimally invasive approach to create an osteotomy and then the holes would be filled with the bioactive substance or substances to support the subchondral bone and or affect a change in the overall alignment of the bone. In the leg for example, a patient may present with a virus alignment of their knee. This results in overloading of the medial compartment of the knee. A BioHTO procedure would involve assessing the patient, planning for correction of small, medium or large angle of malalignment. Specifically, a correction of 1, 2, 3, 4 or more degrees would be affected by making multiple drill holes and then filling them with a biocomposite material that supported or enhanced bone formation as described herein or known in the art. This procedure could also be done in combination with a biochondroplasty. A percutaneous guide would be used in combination with multiple specific drill bits. These specialized drill bits would come in various types based on the need of the patient. One example would be a self-tapping drill bit with a middle section without flutes and then a tapered end. The central portion of the drill bit would contain holes, apertures, and/or openings for release of bioactive substances to support bone. The drill could initially also be cannulated. In some embodiments, the components and compositions as described herein can be assembled into a kit to perform and/or create the methods, procedures, and compositions as described herein.

In some embodiments, the device can include a self-tapping drill bit. The drill bit can have a middle section without flutes and an internal channel disposed along a longitudinal axis of the middle section within the drill bit. In some embodiments, the drill bit can have a tapered end. In some embodiments, the internal channel can have a hollow length extending along at least a part of the longitudinal axis, and the hollow length can have one or two inputs and at least one opening at the tapered end. The at least one opening can allow for the release of a bioactive substance. In some embodiments the hollow length can have multiple openings. In some embodiments, the hollow length can extend along the entire length of the longitudinal axis. In some embodiments, the internal channel can be adapted for delivery of a liquid. In some embodiments, the bioactive substance can be selected from the group consisting of platelet rich plasma (PRP), stem cells, bone marrow cells, bone marrow aspirate, drugs, individual growth factors and synthetic materials. In some embodiments, the awl can be used in combination with the drill bit and introduced into or around the area of where the drill bit is used.

These methods and devices could also be used to treat spinal disc disease in the form of degenerated discs and or degenerated bone and or osteoporotic bone injuries.

Once the area of defective spinal disc tissue is identified, a local, spinal or general anesthetic is administered. The microfracture awl device is inserted into the identified area either under direct vision or via robotic or imaging guidance. Once inside the lesion area, the microfracture awl device is used to create a cavity. The inner cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In some embodiments, the bioactive substance includes PRP. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

An area of defective spinal vertebral bone is initially identified by x-ray, MRI, ultrasound or other imaging modality. The defective spinal vertebral bone may be a compression fracture or other disorder. An area of pain or swelling may also be used to determine the site of a the lesion. Other ways of finding the area of the defect include but are not limited to direct visual inspection and palpation.

Once the area of defective spinal vertebral bone is identified, a local, spinal or general anesthetic is administered. The microfracture awl device is inserted into the identified area either under direct vision or via robotic or imaging guidance. Once inside the lesion area, the microfracture awl device is used to create a cavity. The inner cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In some embodiments, the bioactive substance includes PRP. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure. In some embodiments, the components and compositions can be assembled into a kit to perform and/or create the methods, procedures, and compositions as described herein.

A defective joint such as a knee, shoulder, elbow or ankle is initially identified by x-ray, MRI, ultrasound or other imaging modality. An area of pain or swelling may also be used to determine the site of a the lesion. Other ways of finding the area of the defect include but are not limited to direct visual inspection and palpation.

Once the area of defective joint tissue is identified, a local, spinal or general anesthetic is administered. The microfracture awl device is inserted into the identified area either under direct vision or via robotic or imaging guidance. Once inside the lesion area, the microfracture awl device is used to create a cavity. The cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In some embodiments, the bioactive substance includes PRP. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

A patient presents with tendinitis, tendinosis, or a tendon tear. The defective tendon is initially identified by x-ray, MRI, ultrasound or other imaging modality. An area of pain or swelling may also be used to determine the site of a the lesion. Other ways of finding the area of the defect include but are not limited to direct visual inspection and palpation.

Once the area of defective tendon is identified, a local, spinal or general anesthetic is administered. The microfracture awl device is inserted into the identified area either under direct vision or via robotic or imaging guidance. Once inside the lesion area, the microfracture awl device is used to create a cavity. The cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In some embodiments, the bioactive substance includes PRP. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

A patient presents with osteoporosis or a bone fracture. The defective bone is initially identified by x-ray, MRI, ultrasound or other imaging modality. An area of pain or swelling may also be used to determine the site of a the lesion. Other ways of finding the area of the defect include but are not limited to direct visual inspection and palpation.

Once the area of fractured bone or osteoporosis is identified, a local, spinal or general anesthetic is administered. The microfracture awl device is inserted into the identified area either under direct vision or via robotic or imaging guidance. Once inside the lesion area, the microfracture awl device is used to create a cavity. The cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In some embodiments, the bioactive substance includes PRP. The inner cannula is closed and the awl is used to compress material into the treated area. The awl is removed and the procedure may be repeated multiple times to treat the entire affected area. This procedure may be combined with other procedures to repair the fractured bone. Following the procedure, appropriate clinical and/or imaging modalities are implemented to follow the result of the procedure.

A patient presents with an infection such as a superficial wound or a deep abscess. The infected tissue is initially identified by x-ray, MRI, ultrasound or other imaging modality in combination with physical examination, and/or clinical blood or tissue work to characterize the nature of the disease. An area of pain or swelling may also be used to determine the site of a the infection. Other ways of finding the area of the defect include but are not limited to direct visual inspection and palpation.

Once the infected area is identified, a local, spinal or general anesthetic is administered. The microfracture awl device is inserted into the identified area either under direct vision or via robotic or imaging guidance. Once inside the lesion area, the microfracture awl device is used to create a cavity. The cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, and/or bone marrow cells. In some embodiments, the bioactive substance includes PRP. The inner cannula is closed and the awl is used to compress material into the treated area. The awl is removed and the procedure may be repeated multiple times to treat the entire affected area. This procedure may be combined with other procedures to treat the infection. Following the procedure, appropriate clinical and/or imaging modalities are implemented to follow the result of the procedure.

A patient presents with a melanoma, other skin cancer or skin lesion. The area of the skin lesion is identified visually by physical examination. Clinical evaluation of tissue samples may also be conducted to characterize the disease.

Once the area is identified, an anesthetic is administered, generally a local anesthetic is sufficient. The awl device is inserted into the identified area of skin to penetrate the skin, create a cavity and deliver bioactive substances. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In some embodiments, the bioactive substance includes PRP. The inner cannula is closed and the awl is used to compress material into the treated area. The awl is removed and the procedure may be repeated multiple times to treat the entire affected area. The procedure is combined with appropriate drugs, such as antibiotics or chemotherapy agents. Following the procedure, appropriate clinical and/or imaging modalities are implemented to follow the result of the procedure.

A patient presents with a brain tumor or spinal cord injury. The area of pathological tissue is identified using physical examination or imaging techniques such as x-ray or MRI. Clinical evaluation of tissue or blood samples may also be conducted to characterize the disease.

Once the area is identified, a local, spinal or general anesthetic is administered. The awl device is inserted into the identified bone or nerve sheath to create a cavity and deliver bioactive substances. Either open techniques or endovascular, minimally invasive surgical techniques may be used. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In some embodiments, the bioactive substance includes PRP. The inner cannula is closed and the awl is used to compress material into the treated area. The awl is removed and the procedure may be repeated multiple times to treat the entire affected area. The procedure is combined with appropriate drugs, such as antibiotics or chemotherapy agents. Following the procedure, appropriate clinical and/or imaging modalities are implemented to follow the result of the procedure.

A patient presents with a disease of the eye, ear, nose or throat such as sinus lesions, glaucoma or ear lesions. The area of pathological tissue is identified using physical examination or imaging techniques such as x-ray or MRI. Clinical evaluation of tissue or blood samples may also be conducted to characterize the disease.

Once the area is identified, a local, spinal or general anesthetic is administered. The awl device is inserted into the identified eye, ear, nose or throat tissue to create a cavity and deliver bioactive substances. Either open techniques or endovascular, minimally invasive techniques may be used. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In some embodiments, the bioactive substance includes PRP. The inner cannula is closed and the awl is used to compress material into the treated area. The awl is removed and the procedure may be repeated multiple times to treat the entire affected area. The procedure is combined with appropriate drugs, such as antibiotics or chemotherapy agents. Following the procedure, appropriate clinical and/or imaging modalities are implemented to follow the result of the procedure.

One specific procedure would be to use the compositions and or devices outlined to treat chronic sinusitis. A patient would be identified with the problem and the device would be used to break through chronic sinusitis and deliver bioactive materials to the affected area.

The device may be used for a variety of dental procedures including but not limited to bone grafting, periodontal work or cosmetic work. The area of abnormality is identified using physical examination or imaging techniques such as x-ray or MRI. Once the area is identified, a local, spinal or general anesthetic is administered. The awl device is inserted into the identified tissue to create a cavity and deliver bioactive substances. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In some embodiments, the bioactive substance includes PRP. The inner cannula is closed and the awl is used to compress material into the treated area. The awl is removed and the procedure may be repeated multiple times to treat the entire affected area. The procedure is combined with appropriate drugs, such as antibiotics. Following the procedure, appropriate clinical and/or imaging modalities are implemented to follow the result of the procedure.

The device is used to deliver bioactive material to enhance hair growth. An area of thinning hair growth or baldness is identified using physical examination or imaging techniques such as x-ray or MRI. Once the area is identified, a local anesthetic is administered to numb the area. The awl device is inserted into the identified area of scalp or skin to create a cavity and deliver bioactive substances. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In some embodiments, the bioactive substance includes PRP. A hair follicle is implanted into the cavity created by the awl. The surgical procedure is completed according to standard protocol. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

It will be understood by those of skill in the art that numerous and various modifications can be made without departing from the spirit of the present invention. Therefore, it should be clearly understood that the forms of the present invention are illustrative only and are not intended to limit the scope of the present invention.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

What is claimed is:

1. A method of treating an injury, wear or defect in an individual comprising:
(a) identifying an area of injury, wear or defect;
(b) inserting a device having an awl shaped distal end into the identified area;
(c) creating a cavity in the area using the awl shaped distal end of the device;
(d) attaching a delivery system to an access port for delivery of a bioactive substance;
(e) delivering the bioactive substance through the internal channel and into the identified area;
(f) closing the access port on the device;
(g) compressing the bioactive substance into the identified area; and
(h) removing the device.

2. The method of claim 1, further comprising repeating steps (b) through (h).

3. The method of claim 1, wherein the area of injury, wear or defect comprises a tissue selected from the group consisting of connective tissue, cardiac muscle or tissue, spinal tissue, internal organs, skin tissue, brain tissue, vascular tissue, ocular, ear, nose, and throat tissue.

4. The method of claim 3, wherein the spinal tissue is selected from the group consisting of nerves, spinal cord, disc material and vertebral bodies.

5. The method of claim 3, wherein the internal organ is selected from the group consisting of pancreas, lungs, liver, intestines, and bladder.

6. The method of claim 3, wherein the vascular tissue is selected from the group consisting of veins, arteries and lymphatic tissue.

7. The method of claim 3, wherein the connective tissue is selected from the group consisting of articular cartilage, meniscus cartilage, ligament, tendons, fascia, bone and spinal tissue.

8. The method of claim 1, wherein the bioactive substance is selected from the group consisting of platelet rich plasma, stem cells, bone marrow cells, bone marrow aspirate, drugs, individual growth factors and synthetic materials.

9. The method of claim 8, wherein the bioactive substance is platelet rich plasma, wherein no exogenous activator is added to the PRP prior to delivery into the identified area.

10. The method of claim 9, wherein the PRP comprises platelets obtained from the individual.

11. The method of claim 8, further comprising the step of titrating the PRP to obtain a pH of about 7.3 to 7.5, wherein the titration is performed using a bicarbonate buffer.

12. The method of claim 9, further comprising the step of mixing the PRP substantially simultaneously prior to delivery into the identified area, with one or more ingredients selected from the group consisting of thrombin, epinephrine, collagen, calcium salts, and pH adjusting agents.

13. A device comprising:
a self-tapping drill bit, the drill bit comprises:
a middle section without flutes, the middle section comprising an internal channel disposed along a longitudinal axis within the drill bit; and
a tapered end;
wherein the internal channel comprises a hollow length extending along at least a part of the longitudinal axis, the hollow length having one or two inputs and at least one opening at the tapered end; and
wherein the at least one opening allows for the release of a bioactive sub stance.

14. The device of claim 13, wherein the at least one output comprises multiple openings.

15. The device of claim 13 wherein the hollow length extends along the entire length of the longitudinal axis.

16. The device of claim 13, wherein the internal channel is adapted for delivery of a liquid.

17. The device of claim 13, wherein the bioactive substance is selected from the group consisting of platelet rich plasma (PRP), stem cells, bone marrow cells, bone marrow aspirate, drugs, individual growth factors and synthetic materials.

18. The device of claim 13, further comprising an awl, wherein the awl is configured to be used in combination with the drill bit and introduced into or around the area of where the drill bit is used.

19. A composition for use during an osteotomy procedure comprising:
at least one of a bone supporting substitute, wherein the bone supporting substitute comprises calcium phosphate; and
at least one bioactive substance selected from the group consisting of platelet rich plasma, vitamin D, stem cells, bone marrow cells, bone marrow concentrate, bone marrow aspirate, or adipose concentrate; and
wherein the composition helps to support an osteotomy and affect a change in the alignment of bone.

20. The composition of claim 19, wherein the bone comprises a femur or a tibia.
